(19) United States
(12) Patent Application Publication
(10) Pub. No.: US 2003/0195191 A1
(43) Pub. Date: Oct. 16, 2003

(54) N-sulfonyl hydroxamic acid derivatives as inhibitors of cd23

(76) Inventors: Gordon Burton, King of Prussia, PA (US); Barry Sidney Orlek, King of Prussia, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/275,582

(22) PCT Filed: May 9, 2001
(86) PCT No.: PCT/EP01/05246

(30) Foreign Application Priority Data
May 11, 2000 (GB) 001409.0

(51) Int. Cl. A61K 31/55; A61K 31/454; C 07D 4 1/02

(52) U.S. Cl. 514/217.12; 514/319; 540/604; 546/200; 546/205

(57) ABSTRACT

Compounds of formula (I): wherein R¹ is bicyclyl or heterobicyclyl, R² and R³ are each independently hydrogen, alkyl, alkenyl, alkynyl, aryl, heteroaryl, heterocyclyl, (C1-6)alkylthio, (C2-6)alkenylthio, (C2-6)alkynylthio, aryloxy, arylthio, heterocyclyloxy, heterocyclythio, (C1-6)alkoxy, (C1-6)alkenyloxy, aryl(C1-6)alkoxy, aryl(C1-6)alkylthio, amino, mono- or di-(C1-6)alkylamino, acylamino, sulfonylamino, cycloalkyl, cycloalkenyl, carboxylic acid (C1-6) ester, hydroxy, halogen, carboxamide: CONR⁸R⁹ where R⁸ and R⁹ are independently selected from the group consisting of hydrogen, alkyl, aryl, arylalkyl and heterocyclyl and includes R⁸ and R⁹ as part of a heterocyclyl group, or R² and R³ together form a cyclic alkyl or alkenyl; R⁴ and R⁵ are each independently aryl, heteroaryl, heterocyclyl, alkoxy, alkyl, hydroxy or optionally substituted amino; R⁶ and R⁷ are each hydrogen or together form a fused aryl ring; and m and n are each independently from 0 to 2; with the proviso that when n=1 neither R⁴ nor R⁵ is hydroxy, alkoxy or amino, are useful in the treatment and prophylaxis of conditions mediated by CD23 or TNF.

This invention relates to novel inhibitors of the formation of soluble human CD23 and their use in the treatment of conditions associated with excess production of soluble CD23 (s-CD23) such as autoimmune disease, inflammation and allergy.

CD23 (the low affinity IgE receptor FceRII, Blast 2), is a 45 kDa type II integral protein expressed on the surface of a variety of mature cells, including B and T lymphocytes, macrophages, natural killer cells, Langerhans cells, monocytes and platelets (Delespesse et al, Adv Immunol, 49 [1991] 149-191). There is also a CD23-like molecule on eosinophils (Grangette et al, J Immunol, 143 [1989] 3580-3588). CD23 has been implicated in the regulation of the immune response (Delespesse et al, Immunol Rev, 125 [1992] 77-97). Human CD23 exists as two differentially regulated isoforms, a and b, which differ only in the amino acids at the intracellular N-terminus (Yokota et al, Cell, 55 [1988] 611-618). In man the constitutive a isoform is found only on B-lymphocytes, whereas type b, inducible by IL4, is found on all cells capable of expressing CD23.

Intact, cell bound CD23 (i-CD23) is known to undergo cleavage from the cell surface leading to the formation of a number of well-defined soluble fragments (s-CD23), which are produced as a result of a complex sequence of proteolytic events, the mechanism of which is still poorly understood (Bourget et al J Biol Chem, 269 [1994] 6927-6930). Although not yet proven, it is postulated that the major soluble fragments (Mr 37, 33, 29 and 25 kDa) of these proteolytic events, all of which retain the C-terminal lectin domain common to i-CD23, occur sequentially via initial formation of the 37 kDa fragment (Letellier et al, J Exp Med, 172 [1990] 693-700). An alternative intracellular cleavage pathway leads to a stable 16 kDa fragment differing in the C-terminal domain from i-CD23 (Grenier-Brosette et al, Eur J Immunol, 22 [1992] 1573-1577).

Several activities have been ascribed to membrane bound i-CD23 in humans, all of which have been shown to play a role in IgE regulation. Particular activities include: a) antigen presentation, b) IgE mediated eosinophil cytotoxicity, c) B cell homing to germinal centres of lymph nodes and spleen, and d) downregulation of IgE synthesis (Delespesse et al, Adv Immunol, 49, [1991] 149-191). The three higher molecular weight soluble CD23 fragments (Mr 37, 33 and 29 kDa) have multifunctional cytokine properties which appear to play a major role in IgE production. Thus, the excessive formation of s-CD23 has been implicated in the overproduction of IgE, the hallmark of allergic diseases such as extrinsic asthma, rhinitis, allergic conjunctivitis, eczema, atopic dermatitis and anaphylaxis (Sutton and Gould, Nature, 366, [1993] 421-428).

Other biological activities attributed to s-CD23 include the stimulation of B cell growth and the induction of the release of mediators from monocytes. Thus, elevated levels of s-CD23 have been observed in the serum of patients having B-chronic lymphocytic leukaemia (Sarfati et al, Blood, 71 [1988] 94-98) and in the synovial fluids of patients with rheumatoid arthritis (Chomarat et al, Arthritis and Rheumatism, 36 [1993] 234-242). That there is a role for CD23 in inflammation is suggested by a number of sources. First, sCD23 has been reported to bind to extracellular receptors which when activated are involved in cell-mediated events of inflammation. Thus, sCD23 is reported to directly activate monocyte TNF, IL-1, and IL-6 release (Armant et al, vol 180, J. Exp. Med., 1005-1011 (1994)). CD23 has been reported to interact with the B2-integrin adhesion molecules, CD11b and CD11c on monocyte/macrophage (S. Lecoanet-Henchoz et al, Immunity, vol 3; 119-125 (1995)) which trigger NO2⁻, hydrogen peroxide and cytokine (IL-1, IL-6, and TNF) release. Finally, IL4 or IFN induce the expression of CD23 and its release as sCD23 by human monocytes. Ligation of the membrane bound CD23 receptor with IgE/anti-IgE immune complexes or anti CD23 mAb activates cAMP and IL-6 production and thromboxane B2 formation, demonstrating a receptor-mediated role of CD23 in inflammation.

Because of these various properties of CD23, compounds which inhibit the formation of s-CD23 should have twofold actions of a) enhancing negative feedback inhibition of IgE synthesis by maintaining levels of i-CD23 on the surface of B cells, and b) inhibiting the immunostimulatory cytokine activities of higher molecular weight soluble fragments (Mr 37, 33 and 29 kDa) of s-CD23. In addition, inhibition of CD23 cleavage should mitigate sCD23-induced monocyte activation and mediator formation, thereby reducing the inflammatory response.

TNFα is a pro-inflammatory cytokine which is released from stimulated cells by specific cleavage of a 76-amino acid signal sequence in the inactive precursor to generate the mature form. The cleavage of TNFα has been reported to be carried out by a metalloprotease (Gearing, A. J. H. et al, (1994) Nature 370, 555-557; McGeehan, G. M. et al, (1994) Nature 370, 558-561; Mohler, K. M. et al, (1994) Nature 370, 218-220). Compounds reported to inhibit the cleavage of TNFα by the TNF processing enzyme can be broadly described as matrix metalloprotease inhibitors, particularly of the hydroxamic acid class.

TNFα is induced in a variety of cell types in response to bacteria, endotoxin, various viruses and parasites, so that one physiological function ascribed to TNFα is a contribution to the inflammatory response to acute infection by bacteria, parasites, etc (Dinarello, C. A. (1992) Immunol. 4, 133-145). Overproduction of TNFα has been implicated in disease states such as rheumatoid arthritis, septic shock, Crohn's disease and cachexia (Dinarello, 1992). Inhibition of processing of TNFα to the mature, active form would therefore be beneficial in the treatment of these inflammatory disorders. TNFα may also contribute to the destruction of tissue in autoimmune disease although it is not an initiating factor in these diseases. Confirming the importance of TNFα in rheumatoid arthritis, TNFα antibodies have been shown to reduce the severity of disease in short term studies in rheumatoid arthritis models (Elliott, M. J., et al (1993) Arthrit. Rheum. 12, 1681-1690; Elliott et al (1994) Lancet 344, 1125-1127).

International Patent Application No. WO 97/27174 (Shionogi & Co., Ltd) and International Patent Application number WO 95/35275 (British Biotech Ltd) disclose that certain compounds of formula (A):

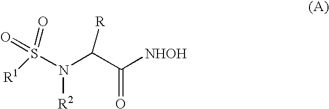

wherein R¹ may be arylalkyl or heteroarylalkyl and R² is hydrogen or an organic substituent are effective inhibitors of metalloproteinases.

International Patent Application No. WO 98/46563 (British Biotech Ltd) discloses that certain compounds of formula (A) above in which R¹ may be phenylalkyl or heteroarylalkyl and R² is hydrogen or an organic substituent are effective inhibitors of matrix metalloproteases.

International Patent Application No. WO 97/18194 (Hoechst) discloses that certain compounds of formula (B):

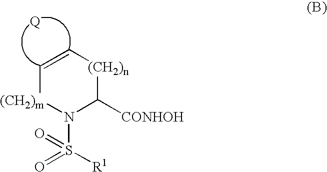

wherein R¹ may be one of a broad range of organic substituents, are effective inhibitors of matrix metalloproteases.

U.S. Pat. No. 5,962,471 (Hoechst) discloses that certain compounds of formula (C):

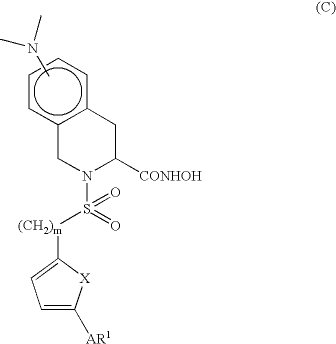

wherein m is 0 to 3 are effective inhibitors of matrix metalloproteases.

European Patent Application No. EP 0 803 505 (Adir) discloses that certain compounds of formula (D):

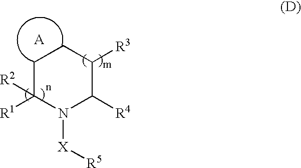

wherein R⁴ may be CONHOH, X may be SO₂ and R⁵ is an optionally substituted alkyl group, cycloalkyl, aryl or heterocyclyl, are effective inhibitors of matrix metalloproteases.

According to the present invention, there is provided a compound of formula (I):

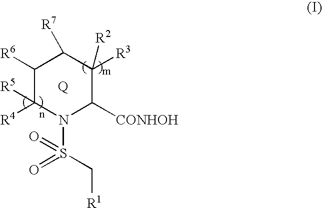

wherein: R¹ is bicyclyl or heterobicyclyl;

R² and R³ are each independently hydrogen, alkyl, alkenyl, alkynyl, aryl, heteroaryl, heterocyclyl, (C1-6)alkylthio, (C2-6)alkenylthio, (C2-6)alkynylthio, aryloxy, arylthio, heterocyclyloxy, heterocyclythio, (C1-6)alkoxy, (C1-6)alkenyloxy, aryl(C1-6)alkoxy, aryl(C1-6)alkylthio, amino, mono- or di-(C1-6)alkylamino, acylamino, sulfonylamino, cycloalkyl, cycloalkenyl, carboxylic acid (C1-6) ester, hydroxy, halogen, carboxamide: CONR⁸R⁹ where R⁸ and R⁹ are independently selected from the group consisting of hydrogen, alkyl, aryl, arylalkyl and heterocyclyl and includes R⁸ and R⁹ as part of a heterocyclyl group, or R² and R³ together form a cyclic alkyl or alkenyl;

R⁴ and R⁵ are each independently aryl, heteroaryl, heterocyclyl, alkoxy, alkyl, hydroxy or optionally substituted amino;

R⁶ and R⁷ are each hydrogen or together form a fused aryl ring; and

m and n are each independently from 0 to 2;

with the proviso that when n=1 neither R⁴ nor R⁵ is hydroxy, alkoxy or amino.

Amino referred to herein in the definition of the R⁴ and R⁵ groups includes amino groups substituted one or more times with (C1-6)alkyl.

Alkyl, alkenyl alkynyl and alkoxy groups referred to herein in the definition of the R², R³, R⁴ and R⁵ groups include straight, branched and cyclic groups containing up to eight carbon atoms, and are optionally substituted by one or more groups selected from the group consisting of aryl, heterocyclyl, (C1-6)alkylthio, (C2-6)alkenylthio, (C2-6)alkynylthio, aryloxy, arylthio, heterocyclyloxy, heterocyclylthio, (C1-6)alkoxy, (C1-6)alkenyloxy, aryl(C1-6)alkoxy, aryl(C1-6)alkylthio, amino, mono- or di-(C1-6)alkylamino, acylamino, sulfonylamino, cycloalkyl, cycloalkenyl, carboxylic acid (C1-6) esters, hydroxy, halogen and carboxamide: CONR⁸R⁹ where R⁸ and R⁹ are independently selected from the group consisting of hydrogen, alkyl, aryl, arylalkyl and heterocyclyl, and includes R⁸ and R⁹ as part of a heterocyclyl group.

Cycloalkyl and cycloalkenyl groups referred to herein in the definition of the R², R³, R⁴ and R⁵ groups include groups having between three and eight ring carbon atoms and are optionally substituted as described hereinabove for alkyl, alkenyl and alkynyl groups.

When used herein in the definition of the R², R³, R⁴, R⁵, R⁶, and R⁷ groups, the term “aryl” includes phenyl. Suitably any aryl group, including phenyl, may be optionally substituted by up to five, preferably up to three substituents. Suitable substituents include halogen, CF₃, OCF₃, CN, (C₁₋₆)alkyl, (C₁₋₆)alkoxy, hydroxy, amino, mono- and di-N-(C1-6)alkylamino, acylamino, acyloxy, carboxy, (C1-6)alkoxycarbonyl, aminocarbonyl, mono- and di-N-(C1-6)alkylaminocarbonyl, mono- and di-N-(C1-6)alkylaminoalkyl, (C1-6)alkylsulfonylamino, aminosulfonyl, (C1-6)alkylthio and (C1-6)alkylsulfonyl. The term “aryl” includes single and fused rings, of which at least one is aromatic, which rings may be unsubstituted or substituted by, for example, up to three substituents as set out above. Each ring suitably has from 4 to 7, preferably 5 or 6, ring atoms.

When used herein in the definition of the R², R³, R⁴ and R⁵ groups the tenn “heteroaryl” suitably includes any heterocyclyl group which incorporates at least one aromatic ring (heterocyclic or carbocyclic).

When used herein in the definition of the R², R³, R⁴ and R⁵ groups the terms “heterocyclyl” and “heterocyclic” suitably include, unless otherwise defined, aromatic and non-aromatic, single and fused, rings suitably containing up to four heteroatoms in each ring, each of which is selected from oxygen, nitrogen and sulphur, which rings, may be unsubstituted or substituted by, for example, up to three substituents. Each ring suitably has from 4 to 7, preferably 5 or 6, ring atoms. A fused heterocyclic ring system may include carbocyclic rings and need include only one heterocyclic ring. Preferably a substituent for a heterocyclyl group is selected from halogen, (C1-6)alkyl, (C1-6)alkoxy, hydroxy, CE₃, OCF₃, CN, amino, mono-and di-N-(C1-6)alkylamino, acylamino, acyloxy, carboxy, (C1-6)alkoxycarbonyl, aminocarbonyl, mono- and di-N-(C1-6)alkylaminocarbonyl, mono- and di-N-(C1-6)alkylaminoalkyl, (C1-6)alkylsulfonylamino, aminosulfonyl, (C1-6)alkylthio and (C1-6)alkylsulfonyl.

When used herein in the definition of the R¹ group “bicyclyl” means fused bicyclic rings suitably containing 4 to 7, preferably 5 or 6 ring atoms in each ring. One ring of the bicyclyl may be saturated or partially saturated. Suitable bicyclyl groups include naphthyl such as 2-naphthyl, tetrahydronaphthyl such as 1,2,3,4-tetrahydronaphthalen-2-yl, and indanyl such as 2-indanyl.

When used herein in the definition of the R¹ group, heterobicyclyl means fused bicyclic aromatic and non-aromatic rings containing up to 4 heteroatoms in each ring, each of which is selected from oxygen, nitrogen and sulphur. Each ring suitably has from 4 to 7, preferably 5 or 6, ring atoms. The fused bicyclic ring system may include one carbocyclic ring and one of the rings may be saturated or partially saturated. Suitable heterobicyclyl groups include benzothiophene such as benzothiophen-5-yl and benzothiophen-6-yl.

Aromatic rings in bicyclyl and heterobicyclyl ring systems may be optionally substituted with up to three substituents. Suitable substituents include fluorine.

In a particular aspect of the invention, R¹ is 2-naphthyl or 5-benzothiophene, and/or R², R³, R⁴ and R⁵ are each independently selected from hydrogen, C₁₋₄alkyl and aryl, and/or R⁶ and R⁷ are each hydrogen or together form a fused phenyl and/or the value of m+n is such that the ring size is 5-7. In this aspect at least one of R² and R³ may be hydrogen and/or at least one of R⁴ and R⁵ may be hydrogen. In a further aspect of the invention, R¹, R², R³, R⁴, R⁵, R⁶, R⁷, m and n are selected from the group consisting of the values ascribed to it in the Examples hereinbelow. Preferably, the compound of formula (I) of the invention is selected from the group consisting of the compounds described in the Examples hereinbelow.

According to a further aspect, the present invention provides the use of a compound of formula (I) for the production of a medicament for the treatment or prophylaxis of disorders such as allergy, allergic asthma, atopic dermatitis and other atopic diseases; inflammatory disorders, and autoimmune disease, in which the overproduction of s-CD23 is implicated.

In a further aspect the invention provides a method for the treatment or prophylaxis of disorders such as allergy, allergic asthma, atopic dermatitis and other atopic diseases; inflammatory disorders, and autoimmune disease, in which the overproduction of s-CD23 is implicated, which method comprises the administration of a compound of formula (I), to a human or non-human mammal in need thereof.

The invention also provides a pharmaceutical composition for the treatment or prophylaxis of disorders such as allergy, allergic asthma, atopic dermatitis and other atopic diseases; inflammatory disorders, and autoimmune disease, in which the overproduction of s-CD23 is implicated which comprises a compound of formula (I) and optionally a pharmaceutically acceptable carrier therefor.

Particular inflammatory disorders include CNS disorders such as Alzheimer's disease, multiple sclerosis, and multi-infarct dementia, as well as the inflammation mediated sequelae of stroke and head trauma.

According to a further aspect, the present invention provides the use of a compound of formula (I) for the production of a medicament for the treatment or prophylaxis of conditions mediated by TNF, including, but not limited to, inflammation, fever, cardiovascular effects, haemorrhage, coagulation and acute phase response, cachexia and anorexia, acute infections, shock states, graft versus host reactions and autoimmune disease.

In a further aspect the invention provides a method for the treatment or prophylaxis of conditions mediated by TNF, which method comprises the administration of a compound of formula (I), to a human or non-human mammal in need thereof.

The invention also provides a pharmaceutical composition for the treatment or prophylaxis of conditions mediated by TNF, which comprises a compound of formula (I) and optionally a pharmaceutically acceptable carrier therefor.

The present inventors have surprisingly found that the compounds of the invention are potent and selective inhibitors of both CD23 processing and TNF processing, whilst having little or no activity as inhibitors of matrix metalloproteases.

It is to be understood that the pharmaceutically acceptable salts, solvates and other pharmaceutically acceptable derivatives of the compound of formula (1) are also included in the present invention.

Salts of compounds of formula (I) include for example acid addition salts derived from inorganic or organic acids, such as hydrochlorides, hydrobromides, hydroiodides, p-toluenesulphonates, phosphates, sulphates, acetates, trifluoroacetates, propionates, citrates, maleates, fumarates, malonates, succinates, lactates, oxalates, tartrates and benzoates.

Salts may also be formed with bases. Such salts include salts derived from inorganic or organic bases, for example alkali metal salts such as sodium or potassium salts, and organic amine salts such as morpholine, piperidine, dimethylamine or diethylamine salts.

The compounds of the invention may be prepared by use of any appropriate conventional method, for example by analogy with the methods disclosed in patent publication EP-A-0 606 046.

Accordingly, a further aspect of the invention provides a process for preparing a compound of formula (1) as defined hereinabove, which process comprises:

(a) deprotecting a compound of formula (II):

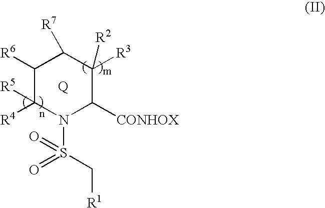

wherein R¹ to R⁷, m and n are as defined hereinabove, and X is a protecting group such as t-butyldimethylsilyl, benzyl or trimethylsilyl, or

(b) reacting a compound of formula (III):

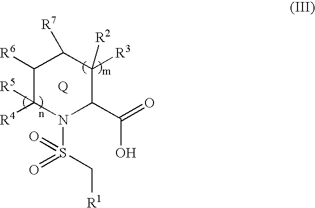

wherein R¹ to R⁷, m and n are as defined hereinabove, with hydroxylamine or a salt thereof, or

(c) converting a compound of formula (I) to a different compound of formula (I) as defined hereinabove.

Compounds of formula (II) and (III) are novel and form a further aspect of the invention.

Compounds of formula (III) can be prepared in accordance with the following reaction scheme, employing a suitable silylating agent, such as BSTFA, to achieve in situ protection of the carboxylic acid group:

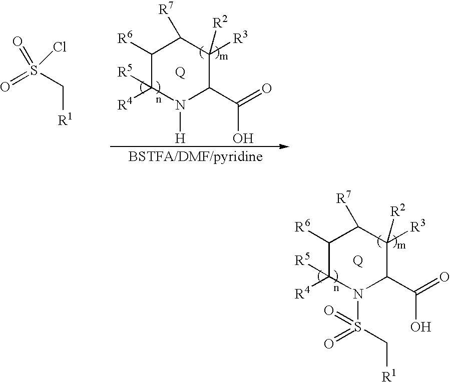

Sulfonylchlorides can be prepared by reacting a compound of formula R¹—CH₂-Z wherein R¹ is as described hereinabove and Z is halogen or methanesulfonate with sodium sulfite to give the corresponding sodium sulfonate, or in the presence of tetra-n-butyl ammonium hydrogen sulfate to give the corresponding tetra-n-butylammonium sulfonate salt. Conversion into the sulfonyl chloride may be achieved using phosphorus oxychloride in acetonitrile and tetrahydrothiophene-1,1-dioxide at elevated temperature (Abdellaoui et al, Synth.Commun.1995, 25(9) 1303). In the case of the tetra-n-butylammonium sulfonate the sulfonyl chloride is prepared using a chlorinating agent such as phosphorus pentachloride or triphosgene.

The starting materials and other reagents are available commercially or can be synthesised by well-known and conventional methods.

The isomers, including stereoisomers, of the compounds of the present invention may be prepared as mixtures of such isomers or as individual isomers. The individual isomers may be prepared by any appropriate method, for example individual stereoisomers may be prepared by stereospecific chemical synthesis starting from chiral substrates or by separating mixtures of enantiomers or mixtures of diastereoisomers using known methods. In a preferred aspect, the invention provides compounds of formula (IA):

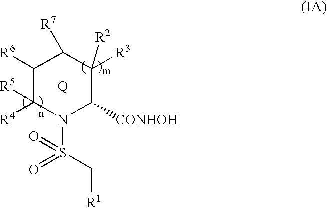

It is preferred that the compounds are isolated in substantially pure form.

As stated herein an inhibitor of the formation of soluble human CD23 has useful medical properties. Preferably the active compounds are administered as pharmaceutically acceptable compositions.

The compositions are preferably adapted for oral administration. However, they may be adapted for other modes of administration, for example in the form of a spray, aerosol or other conventional method for inhalation, for treating respiratory tract disorders; or parenteral administration for patients suffering from heart failure. Other alternative modes of administration include sublingual or transdermal administration.

The compositions may be in the form of tablets, capsules, powders, granules, lozenges, suppositories, reconstitutable powders, or liquid preparations, such as oral or sterile parenteral solutions or suspensions.

In order to obtain consistency of administration it is preferred that a composition of the invention is in the form of a unit dose.

Unit dose presentation forms for oral administration may be tablets and capsules and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulphate.

The solid oral compositions may be prepared by conventional methods of blending, filling or tabletting. Repeated blending operations may be used to distribute the active agent throughout those compositions employing large quantities of fillers. Such operations are of course conventional in the art. The tablets may be coated according to methods well known in normal pharmaceutical practice, in particular with an enteric coating.

Oral liquid preparations may be in the form of, for example, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; non-aqueous vehicles (which may include edible oils), for example almond oil, fractionated coconut oil, oily esters such as esters of glycerine, propylene glycol, or ethyl alcohol; preservatives, for example methyl or propyl p-hydroxybenzoate or sorbic acid; and if desired conventional flavouring or colouring agents.

For parenteral administration, fluid unit dosage forms are prepared utilising the compound and a sterile vehicle, and, depending on the concentration used, can be either suspended or dissolved in the vehicle. In preparing solutions the compound can be dissolved in water for injection and filter sterilised before filling into a suitable vial or ampoule and sealing. Advantageously, adjuvants such as a local anaesthetic, a preservative and buffering agents can be dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspending in the sterile vehicle. Advantageously, a surfactant or wetting agent is included in the composition to facilitate uniform distribution of the compound.

Compositions of this invention may also suitably be presented for administration to the respiratory tract as a snuff or an aerosol or solution for a nebulizer, or as a microfine powder for insufflation, alone or in combination with an inert carrier such as lactose. In such a case the particles of active compound suitably have diameters of less than 50 microns, preferably less than 10 microns for example diameters in the range of 1-50 microns, 1-10 microns or 1-5 microns. Where appropriate, small amounts of other anti-asthmatics and bronchodilators, for example sympathomimetic amines such as isoprenaline, isoetharine, salbutamol, phenylephrine and ephedrine; xanthine derivatives such as theophylline and aminophylline and corticosteroids such as prednisolone and adrenal stimulants such as ACTH may be included.

The compositions may contain from 0.1% to 99% by weight, preferably from 10-60% by weight, of the active material, depending upon the method of administration. A preferred range for inhaled administration is 10-99%, especially 60-99%, for example 90, 95 or 99%.

Microfine powder formulations may suitably be administered in an aerosol as a metered dose or by means of a suitable breath-activated device.

Suitable metered dose aerosol formulations comprise conventional propellants, cosolvents, such as ethanol, surfactants such as oleyl alcohol, lubricants such as oleyl alcohol, desiccants such as calcium sulphate and density modifiers such as sodium chloride.

Suitable solutions for a nebulizer are isotonic sterilised solutions, optionally buffered, at for example between pH 4-7, containing up to 20 mg/ml of compound but more generally 0.1 to 10 mg/ml, for use with standard nebulisation equipment.

An effective amount will depend on the relative efficacy of the compounds of the present invention, the severity of the disorder being treated and the weight of the sufferer. Suitably, a unit dose form of a composition of the invention may contain from 0.1 to 1000 mg of a compound of the invention (0.001 to 10 mg via inhalation) and more usually from 1 to 500 mg, for example 1 to 25 or 5 to 500 mg. Such compositions may be administered from 1 to 6 times a day, more usually from 2 to 4 times a day, in a manner such that the daily dose is from 1 mg to 1 g for a 70 kg human adult and more particularly from 5 to 500 mg. That is in the range of about 1.4×10⁻² mg/kg/day to 14 mg/kg/day and more particularly in the range of about 7×10⁻² mg/kg/day to 7 mg/kg/day.

The following examples illustrate the invention but do not limit it in any way.

Biological Test Methods

Procedure 1: The Ability of Test Compounds to Inhibit the Release of Soluble CD23 was Investigated by Use of the Following Procedure.

RPMI 8866 Cell Membrane CD23 Cleavage Activity Assay:

Plasma membranes from RPMI 8866 cells, a human Epstein-Barr virus transformed B-cell line (Sarfati et al., Immunology 60 [1987] 539-547) expressing high levels of CD23 are purified using an aqueous extraction method. Cells resuspended in homogenisation buffer (20 mM HEPES pH 7.4, 150 mM NaCl, 1.5 mM MgCl2, 1 mM DTT) are broken by N₂ cavitation in a Parr bomb and the plasma membrane fraction mixed with other membranes is recovered by centrifugation at 10,000×g. The light pellet is resuspended in 0.2 M potassium phosphate, pH 7.2 using 2 ml per 1-3 g wet cells and the nuclear pellet is discarded. The membranes are further fractionated by partitioning between Dextran 500 (6.4% w/w) and polyethylene glycol (PEG) 5000 (6.4% w/w) (ref), at 0.25 M sucrose in a total of 16 g per 10-15 mg membrane proteins [Morre and Morre, BioTechniques 7, 946-957 (1989)]. The phases are separated by brief centrifugation at 1000×g and the PEG (upper) phase is collected, diluted 3-5 fold with 20 mM potassium phosphate buffer pH 7.4, and centrifuged at 100,00×g to recover membranes in that phase. The pellet is resuspended in phosphate-buffered saline and consists of 3-4 fold enriched plasma membranes as well as some other cell membranes (e.g. lysosomes, Golgi). The membranes are aliquoted and stored at −80° C. Fractionation at 6.6% Dextran/PEG yields plasma membranes enriched 10-fold.

The fractionated membranes are incubated at 37° C. for times up to 4 hrs to produce fragments of CD23 which are separated from the membrane by filtration in 0.2 micron Durapore filter plates (Millipore) after quenching the assay with a non-selecitve MMP inhibitor, e.g. 5 uM Preparation 1 from WO 95/31457 ([4-(n-Hydroxyamino)-2-(R)-isobutyl-3-(S)-(2-thiophenethiomethyl)succinyl]-(S)-phenylalanine-N-methylamide sodium salt, prepared according to the procedure described in Example 11 of WO 90/05719). sCD23 released from the membrane is determined using the EIA kit from The Binding Site (Birmingham, UK) or a similar one utilising MHM6 anti-CD23 mAb [Rowe et al., Int. J. Cancer, 29, 373-382 (1982)] or another anti-CD23 mAb as the capture antibody in a sandwich EIA. The amount of soluble CD23 made by 0.5 ug membrane protein in a total volume of 50 ul phosphate-buffered saline is measured by EIA and compared to the amount made in the presence of various concentrations of inhibitors. Inhibitors are prepared in solutions of water or dimethylsulfoxide (DMSO) and the final DMSO concentration is not more than 2%. IC50's are determined by curve fitting as the concentration where 50% inhibition of production of sCD23 is observed relative to the difference in sCD23 between controls incubated without inhibitor.

Results

The compounds of the Examples all showed IC₅₀ values <1 μM.

Procedure 2: The Ability of Test Compounds to Inhibit Collagenase was Investigated Using the Following Procedure.

Collagenase Inhibition Assay:

The potency of compounds to act as inhibitors of collagenase was determined by the method of Cawston and Barrett (Anal. Biochem. 99, 340-345, 1979), hereby incorporated by reference, whereby a 1 mM solution of the inhibitor being tested or dilutions thereof, was incubated at 37° C. for 18 h with collagen and human recombinant collagenase, from synovial fibroblasts cloned, expressed and purified from E. Coli, (buffered with 150 mM Tris, pH 7.6, containing 15 mM calcium chloride, 0.05% Brij 35, 200 mM sodium chloride and 0.02% sodium azide). The collagen was acetylated ³H type 1 bovine collagen prepared by the method of Cawston and Murphy (methods in Enzymology 80, 711,1981) The samples were centrifuged to sediment undigested collagen and an aliquot of the radioactive supernatant removed for assay on a scintillation counter as a measure of hydrolysis. The collagenase activity in the presence of 1 mM inhibitor, or dilution thereof, was compared to activity in a control devoid of inhibitor and the results reported as that concentration effecting 50% of the collagenase (IC₅₀).

Results

The compound of Example 2 showed an IC₅₀ value >10 μM.

Preparation of Intermediates

Preparation 1: Naphthalen-2-ylmethanesulfonyl Chloride

Step 1: Sodium naphthalen-2-ylmethanesulfonate—2-Bromomethyl-naphthalene (70 g),was dissolved in dioxan(350 ml) and treated with sodium sulfite (240 g) in water (500 ml). The mixture was heated under reflux for 30 min. On cooling a white solid was obtained which was filtered off, washed with ether and dried to give the subtitle methanesulfonate salt (69 g).

Step 2: Naphthalen-2-ylmethanesulfonyl chloride—To sodium naphthalen-2-ylmethanesulfonate (12 g) in tetrahydrothiophene-1,1-dioxide (96 ml) were added acetonitrile (48 ml) and phosphorus oxychloride (24 ml) and the mixture was heated. When the internal temperature reached 100° C. unreacted starting material was filtered off and the hot filtrate was poured onto ice. A brown solid was filtered off and washed with hexane to give title compound (5.5 g).

Preparation 2: Benzo[b]thiophene-5-methanesulfonyl Chloride

Step 1: 5-Bromomethylbenzo[b]thiophene—A solution containing 5-methylbenzo[b]thiophene (37 g), N-bromosuccinimide (46 g), and tetrachloromethane (400 ml) was refluxed for 4 h, cooled, and filtered. The filtrate was evaporated and the resultant residue crystallised from hexane to give the subtitle compound (40 g).

Step 2: Tetra-n-butylammonium benzo[b]thiophene-5-methanesulfonate—A mixture containing 5-bromomethylbenzo[b]thiophene (40 g), tetra-n-butylammonium hydrogen sulfate (135 g), sodium hydroxide (14 g), sodium sulfite (45 g), dichloromethane (300 ml), and water (300 ml) was stirred vigorously overnight. The organic layer was dried (MgSO₄), evaporated, dissolved in THF (130 ml), re-evaporated, and dissolved again in THF (130 ml). Addition of ether (200 ml) gave the crystalline subtitle compound containing an equimolar amount of tetra-n-butylammonium bromide (132 g).

Step 3: Benzo[b]thiophene-5-methanesulfonyl chloride—A solution of the tetra-n-butylammonium benzo[b]thiophene-5-methanesulfonate from step 2 (30 g) in dichloromethane (150 ml) was added to a cooled suspension of phosphorus pentachloride (8.3 g) in dichloromethane (150 ml) at an internal temperature of −20° C. The solution was warmed to room temperature and maintained at room temperature for 15 min, then filtered through a pad of silicagel washing with ethyl acetate:hexane (1:1). The combined eluates were dissolved in toluene, and the resulting solution again filtered through silica gel, eluting with more toluene. Evaporation of the eluate and crystallisation from hexane gave the title compound (7.5 g). ¹H NMR δ(CDCl₃) 7.95(1H,d,J 8 Hz), 7.94(1H,s), 7.54(1H,d,J 6 Hz), 7.43(1H,d,J 8 Hz), 7.38(1H,d,J 6 Hz), 4.99(2H,s). In like manner was prepared naphthalene-2-methanesulfonyl chloride from 2-bromomethylnaphthalene.

### EXAMPLES

### Example 1

### (R)-1-(Benzo[b]thiophen-5-ylmethanesulfonyl)piperidine-2-carboxylic Acid N-hydroxyamide.

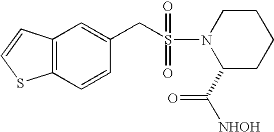

Step 1: (R)-1-(Benzo[b]thiophen-5-ylmethanesulfonyl)piperidine-2-carboxylic acid—(D)-Pipecolinic acid (0.1 g) was taken up in dry DMF (1 ml) and dry pyridine (1 ml) and treated with BSTFA (0.61 ml). The mixture was warmed to 60° C. and left stirring until a clear solution was obtained. The solution was then cooled to 0° C. and benzo[b]thiophene-5-methanesulfonyl chloride (0.19 g) in DMF (1 ml) was added dropwise. The reaction mixture was left stirring at rt for 1 h then treated with methanol (1 ml). After 5 min the solution was passed through a Bond Elut PSA column. After initial elution with methanol the product was eluted with 4% TFA in THF. The THF solution was stripped to dryness to give the subtitle compound as a yellow solid (0.25 g).

Step 2: (R)-1-(Benzo[b]thiophen-5-ylmethanesulfonyl)piperidine-2-carboxylic acid N-hydroxyamide—(R)-1-(Benzo[b]thiophen-5-ylmethanesulfonyl)piperidine-2-carboxylic acid (0.25 g) was taken up in DMF (3 ml). Solid HOAT (0.10 g) and EDC (0.28 g) were added and the mixture was left stirring at rt for 10 min. In a separate flask hydroxylamine hydrochloride (0.28 g) was stirred in DMF (3 ml) was treated with N-methyl morpholine (0.243 ml). The activated acid was then added dropwise to the hydroxylamine solution and left stirring at rt for 2 h. After evaporation of DMF aqueous saturated sodium bicarbonate was added to the residue. The resulting mixture was poured on to a hydromatrix column and the product was obtained by elution with ethyl acetate. Purification by chromatography (Silica gel, step gradient 0-8% methanol/DCM) followed by trituration with ether afforded the title compound as a light brown solid (0.1 g). MS electrospray (−ve ion) 352.8 (M−H⁻); MS electrospray (+ve ion) (MH⁺+NH₃) 371. ¹H NMR δ(DMSO-d₆): 10.6 (1H, s), 8.82 (1H, s), 8.0 (1H, d, J=8 Hz), 7.91 (1H, d, J=0.8 Hz), 7.79 (1H, d, J=5.6 Hz), 7.48 (1H, d, J=5.6 Hz), 7.39 (1H, dd,_J=8.4, 1.6 Hz), 4.54 (1H, d,J=13.6 Hz), 4.40 (1H, d, J=13.6 Hz), 4.17 (1H, d, J=3.6 Hz), 3.30-3.28 (2H, m), 1.82 (1H, m), 1.57 (3H, m), 1.39-1.36 (2H, m).

### Example 2

### (R)-1-(Naphthalen-2-ylmethanesulfonyl)piperidine-2-carboxylic Acid N-hydroxyamide

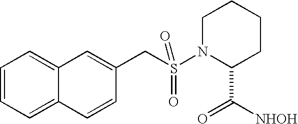

Step 1: (R)-1-(Naphthalen-2-ylmethanesulfonyl)piperidine-2-carboxylic acid—To (D)-Pipecolinic acid (0.177 g) was added DMF (1 ml) and pyridine (1 ml) followed by BSTFA (1 ml). The reaction mixture was warmed to 60° C. After 30 min the solution was cooled to 0° C. and a solution of naphthalene-2-yl-methanesulfonyl chloride (0.3 g) in DMF (1 ml) was added dropwise followed by the addition of NEt₃ (0.174 ml). The reaction mixture was left at rt for 2 h. Aqueous potassium hydrogen sulfate was added to the reaction which was then extracted with ethyl acetate. Evaporation gave the subtitle compound (0.22 g).

Step 2: (R)-1-(Naphthalen-2-ylmethanesulfonyl)piperidine-2-carboxylic acid N-hydroxyamide-O-t-butyldimethylsilyl ether—To (R)-1-(Naphthalen-2-ylmethanesulfonyl)piperidine-2-carboxylic acid (0.2 g) in DCM (5 ml) was added EDC methiodide (0.267 g) and O-t-butyldimethylsilyl hydroxylamine (0.14 g) as a solution in DCM (1 ml). After 2 h the reaction was diluted with ethyl acetate (2 ml) and passed through a short plug of silica. After further elution with with ethyl acetate/hexane (1:1, 10 ml) the eluants were combined and evaporated to give the subtitile compound (0.25 g).

Step 3: (R)-1-(Naphthalen-2-ylmethanesulfonyl)piperidine-2-carboxylic acid N-hydroxyamide—(R)-1-(Naphthalen-2-ylmethanesulfonyl)piperidine-2-carboxylic acid N-hydroxyamide-O-t-butyldimethylsilyl ether (0.23 g) in THF (1.5 ml) was treated with tetrabutylammonium fluoride (0.8 ml of a IM solution in THF). After 40 min the reaction was evaporated, diluted with ethyl acetate and washed with saturated sodium bicarbonate and brine. The solution was passed down an SCX column and evaporated. The residue was triturated with hexane before crystallisation from toluene/ether to give the titled compound (0.114 g). MS electrospray (−ve ion) 347 (M−H⁻); MS electrospray (+ve ion) (M⁺+NH₃) 366. ¹H NMR δ(CD₃OD): 7.4-8.0 (7H, m), 4.6 (1H, d, J=13.6 Hz), 4.47 (1H, d, J=13.6 Hz), 4.32 (1H, m), 3.4-3.64 (2H, m), 1.88-2.05 (1H, m), 1.3-1.8 (5H, m).

The compounds of the following examples were prepared by the procedures described in Example 1 and Example 2. ¹H NMR and mass spectra were consistent with the proposed structures.

### Example 3

### (R)-2-(Naphthalen-2-ylmethanesulfonyl)-1,2,3,4-tetrahydroisoquinolin-3-carboxylic Acid N-hydroxyamide

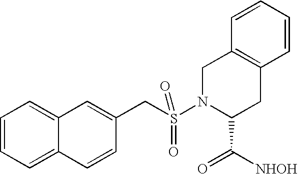

### Example 4

### (R)-2-(Benzo[b]thiophen-5-ylmethanesulfonyl)-1,2,3,4-tetrahydroisoquinolin-3-carboxylic Acid N-hydroxyamide

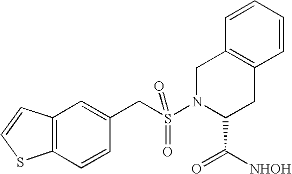

Abbreviations

Bond Elut PSA bonded silica supplied by Varian

BSTFA—Bis trimethylsilyltrifluoroacetamide

DCM—Dichloromethane

DMF—N,N-Dimethylformamide

EDC—1-(3-dimethylaminopropyl)-3-ethylcarbodiimide hydrochloride

EDC—methiodide-1-(3-dimethylaminopropyl)-3-ethylcarbodiimide methiodide

HOAT—1-Hydroxy-7-azabenzotriazole

rt—Room temperature

SCX—Bonded silica cation exchange support supplied by Varian

TBAF—Tetra-n-butylammonium fluoride

TFA—Trifluoroacetic acid

THF—Tetrahydrofuran

1. A compound of formula (I): 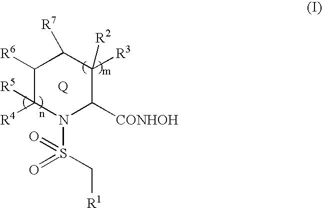
wherein: R¹ is bicyclyl or heterobicyclyl;
R² and R³ are each independently hydrogen, alkyl, alkenyl, alkynyl, aryl, heteroaryl, heterocyclyl, (C1-6)alkylthio, (C2-6)alkenylthio, (C2-6)alkynylthio, aryloxy, arylthio, heterocyclyloxy, heterocyclythio, (C1-6)alkoxy, (C₁-6)alkenyloxy, aryl(C₁-6)alkoxy, aryl(C1-6)alkylthio, amino, mono- or di-(C1-6)alkylamino, acylamino, sulfonylamino, cycloalkyl, cycloalkenyl, carboxylic acid (C1-6) ester, hydroxy, halogen, carboxamide: CONR⁸R⁹ where R⁸ and R⁹ are independently selected from the group consisting of hydrogen, alkyl, aryl, arylalkyl and heterocyclyl and includes R⁸ and R⁹ as part of a heterocyclyl group, or R² and R³ together form a cyclic alkyl or alkenyl;
R⁴ and R⁵ are each independently aryl, heteroaryl, heterocyclyl, alkoxy, alkyl, hydroxy or optionally substituted amino;
R⁶ and R⁷ are each hydrogen or together form a fused aryl ring; and
m and n are each independently from 0 to 2;
with the proviso that when n=1 neither R⁴ nor R⁵ is hydroxy, alkoxy or amino:

2. A compound of formula (IA): 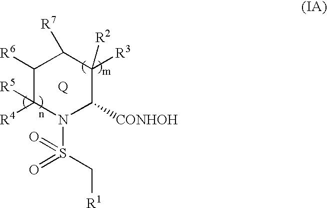
wherein: R¹ is bicyclyl or heterobicyclyl;
R² and R³ are each independently hydrogen, alkyl, alkenyl, alkynyl, aryl, heteroaryl, heterocyclyl, (C1-6)alkylthio, (C2-6)alkenylthio, (C2-6)alkynylthio, aryloxy, arylthio, heterocyclyloxy, heterocyclythio, (C1-6)alkoxy, (C1-6)alkenyloxy, aryl(C1-6)alkoxy, aryl(C1-6)alkylthio, amino, mono- or di-(C1-6)alkylamino, acylamino, sulfonylamino, cycloalkyl, cycloalkenyl, carboxylic acid (C1-6) ester, hydroxy, halogen, carboxamide: CONR⁸R⁹ where R⁸ and R⁹ are independently selected from the group consisting of hydrogen, alkyl, aryl, arylalkyl and heterocyclyl and includes R⁸ and R⁹ as part of a heterocyclyl group, or R² and R³ together form a cyclic alkyl or alkenyl;
R⁴ and R⁵ are each independently aryl, heteroaryl, heterocyclyl, alkoxy, alkyl, hydroxy or optionally substituted amino;
R⁶ and R⁷ are each hydrogen or together form a fused aryl ring; and
m and n are each independently from 0 to 2;
with the proviso that when n−1 neither R⁴ nor R⁵ is hydroxy, alkoxy or amino.

3. A compound according to claim or claim 2 wherein R¹ is 2-naphthyl or 5-benzothiophene, and/or R², R³, R⁴ and R⁵ are each independently selected from hydrogen, C₁₋₄alkyl and aryl, and/or R⁶ and R⁷ are each hydrogen or together form a fused phenyl and/or the value of m+n is such that the size of ring system Q is 5-7 carbon atoms.

4. A compound selected from the group consisting of:
(R)-1-(Benzo[b]thiophen-5-ylmethanesulfonyl)piperidine-2-carboxylic acid N-hydroxyamide;
(R)-1-(Naphthalen-2-ylmethanesulfonyl)piperidine-2-carboxylic acid N-hydroxyamide;
(R)-2-(Naphthalen-2-ylmethanesulfonyl)-1,2,3,4-tetrahydroisoquinolin-3-carboxylic acid N-hydroxyamide; and
(R)-2-(Benzo[b]thiophen-5-ylmethanesulfonyl)-1,2,3,4-tetrahydroisoquinolin-3-carboxylic acid N-hydroxyamide.

5. Use of a compound according to any preceding claim for the production of a medicament for the treatment or prophylaxis of disorders in which the overproduction of s-CD23 is implicated.

6. A method for the treatment or prophylaxis of disorders in which the overproduction of s-CD23 is implicated, which method comprises the administration of a compound according to any one of claims 1 to 4 to a human or non-human mammal in need thereof.

7. A pharmaceutical composition for the treatment or prophylaxis of disorders in which the overproduction of s-CD23 is implicated which comprises a compound according to any one of claims 1 to 4 and optionally a pharmaceutically acceptable carrier therefor.

8. Use of a compound according to any one of claims 1 to 4 for the production of a medicament for the treatment or prophylaxis of conditions mediated by TNF.

9. A method for the treatment or prophylaxis of conditions mediated by TNF, which method comprises the administration of a compound according to any one of claims 1 to 4 to a human or non-human mammal in need thereof.

10. A pharmaceutical composition for the treatment or prophylaxis of conditions mediated by TNF, which comprises a compound according to any one of claims 1 to 4 and optionally a pharmaceutically acceptable carrier therefor.

11. A process for preparing a compound according to any one of claims 1 to 3 which process comprises:
(a) deprotecting a compound of formula (II): 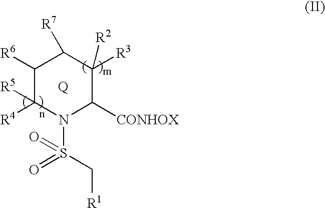
wherein R¹ to R⁷, m and n are as defined hereinabove, and X is a protecting group such as t-butyldimethylsilyl, benzyl or trimethylsilyl, or
(b) reacting a compound of formula (III): 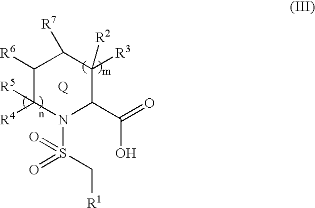
wherein R¹ to R⁷, m and n are as defined hereinabove, with hydroxylamine or a salt thereof, or
(c) converting a compound of formula (I) to a different compound of formula (I) as defined hereinabove.

12. A compound of formula (II): 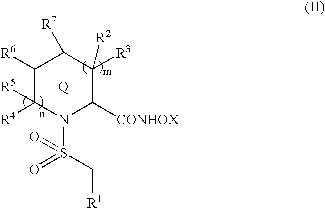
wherein R¹ to R⁷, m and n are as defined hereinabove, and X is a protecting group.

13. A compound of formula (III): 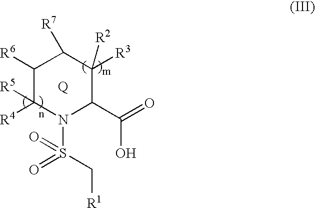
wherein R¹ to R⁷, m and n are as defined hereinabove.
